(19) United States
(12) Patent Application Publication
(10) Pub. No.: US 2003/0225404 A1
(43) Pub. Date: Dec. 4, 2003

(54) Low power electrode

(76) Inventors: James B. Logan, Seminole, FL (US); Phillip J. Berman, Clearwater, FL (US)

Correspondence Address: GENE WARZECHA, LINVATEC CORPORATION, 11311 CONCEPT BOULEVARD, LARGO, FL, 33773

(21) Appl. No.: 10/157,164

(22) Filed: May 29, 2002

(51) Int. Cl. A61B 18/18

(52) U.S. Cl. 606/45

(57) ABSTRACT

A monopolar electrode for use with a standard electrosurgical pencil and generator suitable for performing tissue ablation at relatively low power levels. The electrode has an active portion at its distal tip, the active portion being partially or totally covered by a dielectric material to create high power densities sufficient to cause the electrode sparking necessary for tissue ablation. Portions of the electrode other than the active portion are surrounded by an insulating material to enable transmission of electromagnetic energy only through the active portion.

### BACKGROUND OF THE INVENTION

1. Field of the Invention

The invention relates to the ablation of tissue during electrosurgical procedures. More particularly, the invention relates to tissue ablation by electrosurgical devices in a fluid environment during arthroscopic procedures.

2. Description of the Prior Art

Electrosurgical procedures are commonly performed in either a monopolar mode, using a probe having an active electrode placed adjacent tissue to be operated upon, with a return or common electrode placed externally on the patient's body, or a bipolar mode where both active and return electrodes are on the same probe. The procedures are used to cut, coagulate or ablate tissue, these different functions accomplished by applying different energy waveforms and/or power levels to the electrodes.

The present invention is related to the properties of electrical conductivity of a monopolar or bipolar endoscopic, preferably arthroscopic electrode, in a conducting fluid environment. Electrical (radio frequency) energy is passed through the electrode from a conventional electrosurgical generator, out the active tip of the electrode, through the surrounding medium and back to the return electrode (either near the active electrode or at a remote grounding pad). In practice, an electrode powered at a given level must “fire” or generate sparks (i.e. arcing) in order to cut or ablate tissue. That is, the power and energy density emanating from the electrode must be sufficiently high to cause arcing from the electrode to the tissue. It is known that the energy density may be made sufficiently high by moving the electrode closer to or in contact with the tissue to be ablated, by increasing to a predetermined level the amount of power coming from the generator into the electrode or by changing the geometry of the electrode (for example, sharper edges produce higher energy densities).

It is sometimes preferable to have the electrode generate sparks before it is brought into the vicinity of tissue but, prior to the present invention, the only known way to do this for a given electrode geometry was to increase the power level. However, increasing power may produce rapid and unintended tissue ablation or charring before the surgeon is able to get the process under control. Achieving sparking while the electrode is relatively far from the tissue allows the surgeon to control the process before affecting tissue.

Power density on the surface of an electrode (monopolar or bipolar) is somewhat dependent on the conductivity of tissues or fluids in contact with the electrode. The fluids used in arthroscopic electrosurgery are highly conductive and produce non-uniform current (and power) density at the electrode surface. As shown in U.S. Pat. No. 6,149,646 (West, Jr. et al.), assigned to the assignee hereof and incorporated by reference herein, maximizing the power density over large enough surfaces facilitates tissue ablation, and the invention described in the West patent facilitates the proper power density over large enough surfaces of monopolar electrodes at power levels lower than those commonly required by bipolar tissue ablation devices.

For an electrosurgical instrument working in a space filled with conductive fluid, such as during an arthroscopic procedure, current density is known to be higher at the edges of the electrode than on its broader or flatter surfaces. When sufficient power is supplied, the current density at the edge of an electrode in this environment is sufficient to raise the temperature of the ambient fluid thereby making it more conductive. The increased current flow due to this increased conductivity further raises the fluid temperature, which increases the conductivity, which increases the current flow, etc. This continues until the fluid at the electrode edge begins to form a gas phase due to boiling and a luminous discharge becomes visible due to localized arcing. It is believed that the high current density discharge and intense heat at the electrode edge actually perform the ablation. Similarly, bringing the edge of the instrument into contact or sufficiently close proximity with tissue will facilitate initiation of discharge from the edge of the electrode nearest the tissue. If sufficient power is supplied after such high-density discharge is initiated, the instrument can be withdrawn slightly from the tissue while maintaining the high-density discharge at the electrode edge. This phenomenon is well known to surgeons using conventional monopolar electrosurgical instruments.

It is desirable to minimize the application of energy to electrodes used in electrosurgery while maintaining or increasing power density at the active electrode. Electrodes capable of properly operating at low power levels can be used with smaller, less expensive electrosurgical generators and can be easily used by surgeons with less risk of inadvertently harming the patient.

The present invention provides another means for increasing the energy density of an electrode by selectively impeding the electrical field of the energy emanating from the active electrode to concentrate it and create a sufficiently high energy density to allow the electrode to generate sparks at a power setting lower than would otherwise be required.

It is accordingly an object of this invention to produce an electrosurgical tissue ablator suitable for use with conventional electrosurgical generators in a monopolar mode.

It is also an object of this invention to produce a monopolar tissue ablator capable of ablating relatively large volumes of tissue at relatively low power levels.

It is also an object of this invention to produce a monopolar electrode capable of producing high power density levels sufficient for tissue ablation while being driven by relatively low power levels, preferably less than approximately 50 watts.

It is also an object of this invention to produce a monopolar electrode capable of producing tissue ablation within a surgical field filled with conductive fluid.

It is yet another object of this invention to produce electrosurgical electrodes capable of operating properly at power levels lower than those required by known electrodes.

It is still another object of this invention to produce an electrode having a spark initiator which enables the electrode in a conductive fluid to fire at relatively low power levels even which it is not near tissue.

### SUMMARY OF THE INVENTION

These and other objects are achieved by the preferred embodiment disclosed herein which employs an elongated, solid, electrically conductive rod having a distal end terminating in a conducting electrode tip which has an active portion serving as a source of radio frequency electromagnetic energy. The rod is insulated along its length up to the distal end. A second insulating material, having a higher dielectric property is situated over a predetermined part of the active portion and interposed between it and the tissue to be treated.

Another aspect of the invention is the method of using an electrosurgical electrode. The method comprises the steps of providing an electrosurgical electrode having an active portion thereof from which electromagnetic energy may emanate and interposing a dielectric material between said active portion of said electrode and the tissue to be treated.

### BRIEF DESCRIPTION OF THE DRAWINGS

FIG. 1 shows a prior art monopolar electrode situated at the tip of an electrosurgical pencil.

FIG. 2 shows a monopolar electrode constructed in accordance with the principles of this invention.

FIG. 3 is a side elevation view of the distal end of the electrode of FIG. 2.

FIG. 4 is a cross-sectional view of FIG. 3.

FIG. 5 is a view of FIG. 3 with the insulation sleeve removed.

FIG. 6 is a front perspective view of FIG. 3.

FIG. 7 is a right side elevation view of FIG. 3.

FIG. 8 is a front perspective view of an alternate embodiment of an electrode constructed in accordance with the principles of this invention.

### DESCRIPTION OF THE PREFERRED EMBODIMENT

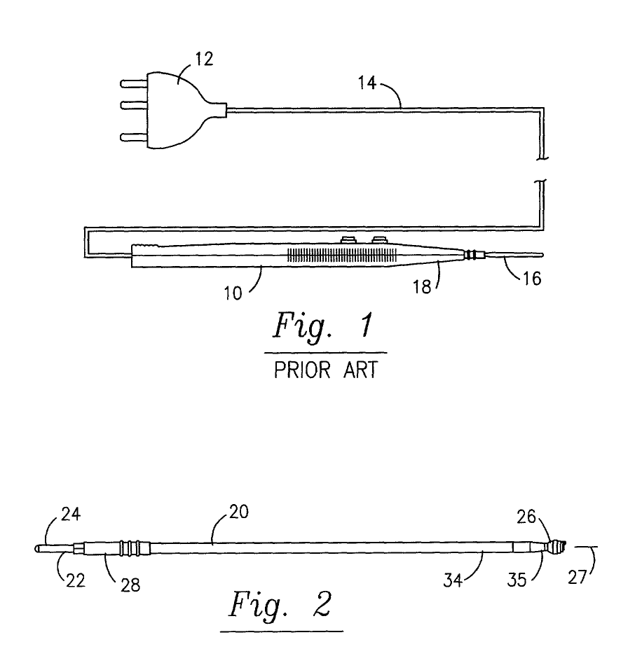

Referring now to FIG. 1, there is shown a conventional monopolar electrosurgical pencil 10 connected to a plug 12 via a power cord 14. Pencil 10 is a conventional unit which is designed to be plugged into a conventional electrosurgical generator (not shown) and is adapted to receive a variety of electrodes 16 in its distal end 18.

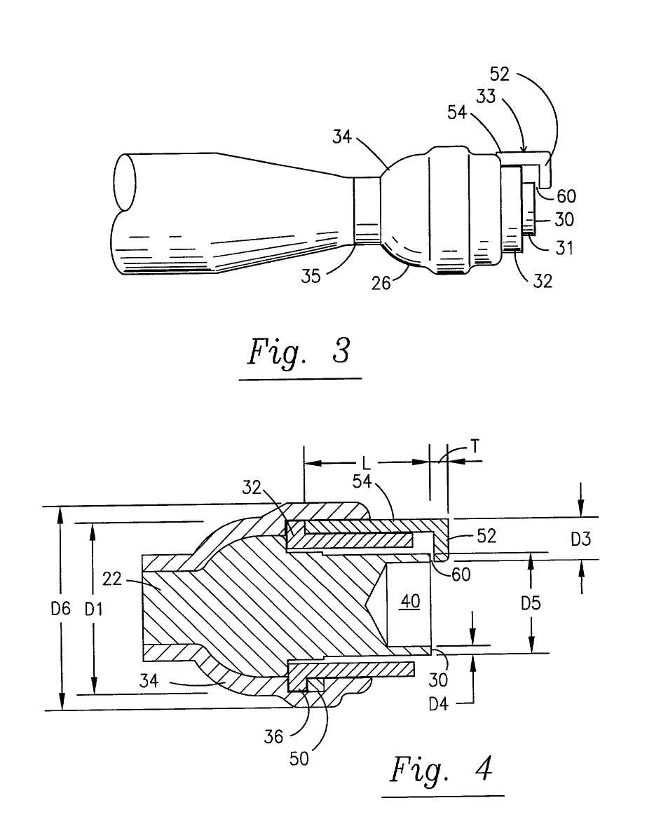

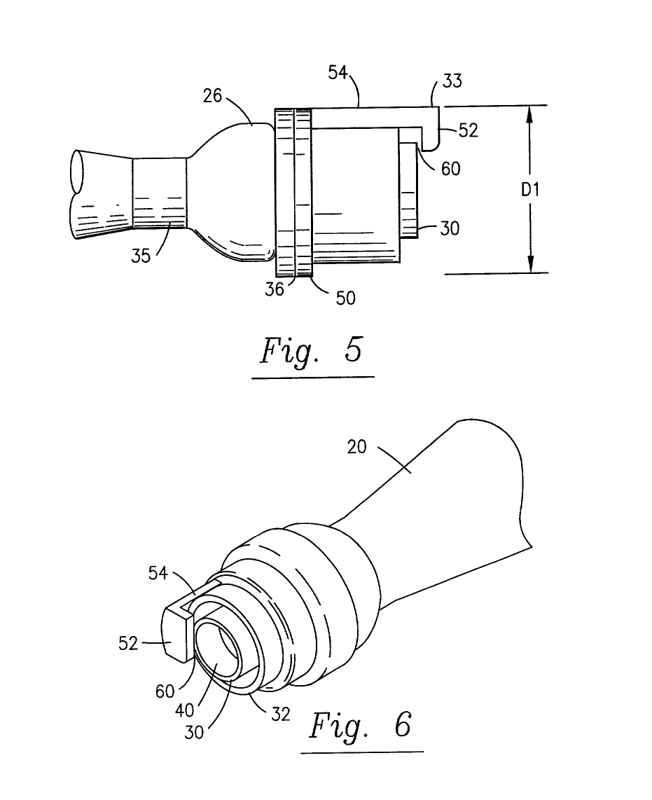

One type of electrode 16 which may be used is shown in FIGS. 2-6 as ablation electrode 20 constructed in accordance with the principles of this invention. Electrode 20 comprises an electrically conductive cylindrical rod 22 having a proximal end 24, a distal end 26 and an axis 27. Rod 22 is secured to and extends through a conventional polymeric hub 28 adapted to facilitate connecting the electrode to pencil 10. Rod 22 is reduced in diameter at neck 35 to facilitate bending of end 26 relative to axis 27. The distal end 26, best seen in FIG. 3, has an electrode tip provided with an active electrode 31 in the form of an annular, distally facing electrode surface, cylindrical rim 30, ceramic insulator 32 and spark initiator 33. Rim 30 surrounds a recess 40 and is a feature of a preferred embodiment of this invention. It will be understood, however, that other configurations of active electrode 31 could be used in the invention. For example, the distally facing electrode surface could be solid and planar or could be a plurality of planar elements such as parallel ribs, concentric circles or even a “checkerboard” pattern created by the intersections of two sets of parallel cut arranged orthogonally to each other. Polymeric insulating sleeve 34 extends longitudinally along electrode 20 and covers all but the distal-most and proximal-most parts of the exposed external surfaces of rod 22. Sleeve 34 also covers the adjacent distal exterior surface of hub 28 and the proximal exterior surface of ceramic sleeve 32, as well as predetermined proximal portion of spark initiator 33. The structure of the electrode tip (not including the spark initiator) is essentially as taught in the aforementioned U.S. Pat. No. 6,149,646 which is incorporated by reference herein.

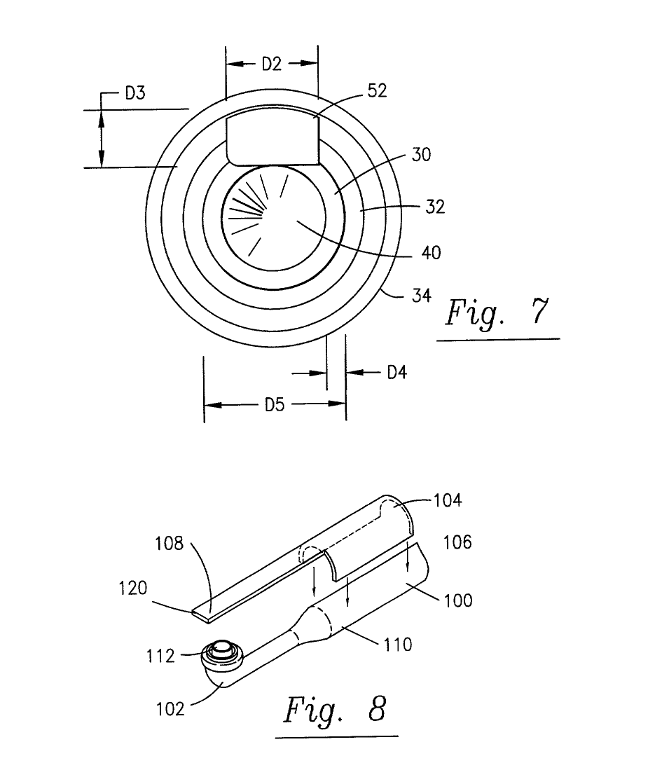

As best seen in FIGS. 3 and 4, coaxial ceramic insulator sleeve 32 is provided with a circular flange 36 and spark initiator 33 has a base ring 50 which rests on (and is preferably secured to) flange 35. Both flange 35 and base ring 50 have the same outside diameter D1. Spark initiator 33 further comprises a distal cap 52 and a longitudinally extending connecting arm 54 joining base 50 and cap 52. Connecting arm 54 lies on the cylindrical surface of sleeve 32 and serves to position cap 52 at a predetermined longitudinal position relative to electrode rim 30 to produce a gap 60. Cap 52 covers a predetermined portion of active electrode 31. As used herein, the term “cover” means that when active electrode 31, e.g. rim 30, is viewed by an observer, as for example, in plan view as shown in FIG. 7, the cap 52 is interposed between that predetermined portion and the observer and hides that portion from view. The term “cover” may be used to describe the relationship between cap 52 and rim 30 where both the rim and the cap lie parallel to each other on opposite sides of gap 60. The term “cover” may also be used to describe other configurations where a cap like cap 52 could be situated near enough to an active electrode to achieve the effects taught herein.

The particular structure of the spark initiator 33 is dependent upon how cap 52 is desired to be shaped and secured so as to produce gap 60. In a first embodiment of an example of this invention, as shown in the drawings, it has been found that on an electrode having a diameter D1=0.094 inches (2.388 mm) cap 52 could be formed as a transverse projection having a length D3 and a wide D2 at the distal end of connecting arm 54, as best seen in FIGS. 3 and 6. Such a structure covers a predetermined portion of the active electrode 31, which in this case is rim 30. In this example, width D2 ranged from 0.010 inches (0.254 mm) to 0.020 inches (0.508 mm), transverse length D3 was approximately equal to the dimension across ceramic sleeve 32 and rim 30 (i.e. approximately 0.025 inches (0.635 mm)), the thickness D4 of rim 30 was 0.004 inches (0.102 mm), the diameter D5 of rim 30 was 0.054 inches (1.37 mm). The gap 60 between the surface of electrode rim 30 and cap 52 is between zero inches (0.0 mm) and 0.010 inches (0.254 mm), preferably between 0.002 inches (0.051 mm) and 0.005 inches (0.127 mm). Further dimensions of this example comprise overall diameter D6 equal to 0.114 inches (2.896 mm) which is the diameter of electrode distal end 26 with insulating sleeve 34, length L of connecting arm which is 0.080 inches (2.03 mm) and thickness T of cap 52 which is 0.010 inches (0.254 mm). With these dimensions and a power input of 35-40 watts in the coag mode, electrode 20 was able to fire in saline without any need to bring tissue into close proximity to rim 30. Once the spark was formed, the power could be increased before or after beginning to cut or ablate tissue. In the foregoing example, the electrode rod 22 was made of stainless steel approximately 5.12 inches (130 mm) long and spark initiator 33 was made of a non-conducting insulating material of high dielectric strength. While it is believed that an insulating material could be used to make spark initiator 33, this material must be rigid enough to maintain gap 60 (at least until the electrode fires) and must have a dielectric strength sufficiently high to prevent the breakthrough of energy across cap 52 (at least until the electrode fires). It is believed that high dielectric strength is the most significant characteristic of spark initiator 33 and, therefore, the terms “dielectric” and “insulator” may be used interchangeably with respect to descriptions of the spark initiator.

In a second embodiment of the foregoing example, the overall size of the electrode distal end was increased, although the same general proportions were maintained. Thus, in the second embodiment D1=0.130 inches (3.30 mm), D2=0.010 inches (0.254 mm) to 0.020 inches (0.508 mm), D3=0.032 inches (0.813 mm), D4=0.006 inches (0.152 mm), D5=0.078 inches (1.981 mm), D6=0.150 inches (3.81 mm), L=0.069 inches (1.753 mm) and T=0.011 inches (0.279 mm).

While electrode 20 may be made in various sizes, the relative proportions disclosed herein facilitate its operation. It will be understood, however, that changes in materials and power levels may alter the relative proportions. While an electrode constructed with these dimensions has been found to ablate tissue at input power levels on the order of 30-50 watts, it will be understood that satisfactory ablation may occur at lower power levels with dimensional changes in the electrode. Additionally, some users may prefer to operate at power levels greater than 50 watts for certain tissue. In view of the above, the intended power range for the invention is deemed to be a range on the order of 0-100 watts, preferably 30-50 watts.

In one example, the dielectric material used was a polymeric material (high dielectric strength) sold under the trademark C-FLEX®. This material was placed over the distal metallic tip of a monopolar Ultrablator® electrode. It was found that while an uncovered Ultrablator® electrode would, under normal use in a conductive saline environment and not near tissue, generate continuous sparks at 100 watts in the coag mode and 140 watts in the cut mode, an Ultrablator® with its tip covered to some extent by the C-FLEX® polymer would generate continuous sparks at 45 watts in the coag mode and 95 watts in the cut mode. As used herein, “cut mode” is defined as a continuous sinusoidal or generally sinusoidal waveform of radio frequency energy and the “coag (coagulation) mode” is defined as a non-continuous sinusoidal or generally sinusoidal waveform of energy having much higher amplitudes for a given power level than the cut mode. While the electrode was made to fire at low power levels when the dielectric material touched the electrode rim surface, increasing the clearance gap between the C-FLEX® polymer sleeve and the electrode tip produced sparks at higher power settings. That is, there is a correlation between the gap between the dielectric and the electrode and the power required to fire. As the dielectric was moved away from the electrode tip, it was found necessary to increase the power to achieve a continuous spark.

It is not necessary for the dielectric to cover the entire electrode tip. In another experiment only a portion of the electrode was covered. Nevertheless, a continuous spark was generated with the power settings in the 65 to 75 watt range, even though the electrode tip was not in the vicinity of tissue.

It is noted that with the embodiment of FIGS. 2-7 one could use the electrode in the straight configuration shown or one could bend distal end 26 at neck 35 to produce any degree of bend desired. The spark initiator is situated distally of neck 35 and follows the electrode rim 30. As noted by FIG. 7, other embodiments of the electrode may be made with prebent ends and with the spark initiator shaped appropriately to be attached to the electrode body while simultaneously covering a predetermined part of the active portion of the electrode.

It is believed that spark initiator 33 facilitates the firing of the electrode because the material of the spark initiator has a high enough dielectric strength to concentrate the energy from the electrode and prevent the breakdown of cap 52 at the power levels experienced by electrode 20. It is believed that this dielectric strength enables cap 52 to concentrate the energy emanating from the covered portion of the active electrode 31 (e.g. rim 30) so that the conductive fluid adjacent to the cap 52 or trapped between the covered portion and cap 52 is heated to sparking temperature more quickly than if cap 52 was not used. Once the spark is initiated in or at the edges of gap 60, the spark spreads rapidly all around the active electrode 31 (e.g. rim 30). The C-FLEX® material used has a dielectric strength of approximately 500 volts/mil (20×10³ V/mm). The material has a lower dielectric strength than that of sheath 34, which preferably has a dielectric strength of 1500 V/mil, but it has a resistance to thermal breakdown that enables it to withstand the operating temperatures of the electrode.

As shown in FIG. 7 the distal end of rod 22 may be bent relative to the axis of the rod at 900 to produce alternate electrode embodiment 100. It will be understood that the distal end could be bent to any other suitable angle to produce additional embodiments (not shown). Ablation electrode 100 may be constructed as shown in aforementioned U.S. Pat. No. 6,149,646 and modified as taught herein. Thus, electrode 100 has secured to its distal end 102 a spark initiating sleeve 104 having a body 106 and a distal extension 108. Body 106 is secured to electrode shaft 110 so that extension 108 is situated over a predetermined portion of the electrode tip 112. It will be understood that distal extension 108 may be produced with a variety of end configurations depending upon the portion of tip 112 that is desired to be covered.

The method of using the electrodes disclosed herein comprises the steps of applying to the electrodes a relatively low power level of 30 to 40 watts and initiating the spark prior to contacting tissue. The method further comprises ablating tissue at this low power level and then increasing the power level to achieve the desired effect.

The invention is most useful when applied to monopolar electrodes which work in conjunction with a return electrode situated at a site well removed from the monopolar electrode. However, the invention may also be utilized with bipolar electrodes. As used herein, the term “bipolar electrodes” refers to electrodes which have an active electrode situated on a probe with a return electrode in close proximity to the active electrode, preferably on the same probe. In bipolar electrodes the arcing occurs from the active electrode to the return electrode. The term “close proximity” as used herein means sufficiently close so as to be at the same surgical site within the body in order to be able to receive the energy emanating from the active electrode.

While electrodes, both monopolar and bipolar, are usually made in the form of elongated probes in which the active electrode is at the distal end, the invention will work with any configuration. For example, the active electrode could be situated at any point along a supporting member.

While the spark initiator 33 and insulating sleeve 34 are shown as separate and distinct elements, it will be understood that, given the right materials it would be possible to make these elements as one homogeneous element.

It will be understood by those skilled in the art that numerous improvements and modifications may be made to the preferred embodiment of the invention disclosed herein without departing from the spirit and scope thereof.

What is claimed is:

1. An electrode for treating tissue, said electrode for use with an electrosurgical pencil connected to an electrosurgical generator comprising:
an electrically conductive rod having a distal end and a proximal end with an axis, said proximal end adapted to be connected to said electrosurgical pencil, said distal end terminating in a conducting electrode tip, said electrode tip comprising an active portion for serving as a source of radio frequency electromagnetic energy;
a first insulating member situated about said rod and a predetermined portion of said electrode tip; and
a second insulating member covering a predetermined portion of said active portion of said electrode tip.

2. An electrode according to claim 1 wherein said second insulating member completely covers said active portion.

3. An electrode according to claim 1 further comprising a return electrode in close proximity to said electrode tip.

4. An electrode according to claim 1 wherein said first insulating member is unitarily formed with said second insulating member.

5. An electrode according to claim 1 wherein said second insulating tubular member has a dielectric strength greater than or equal to 500 volts/mil.

6. An electrode according to claim 1 wherein said second insulating tubular member has a dielectric strength between 500 and 1500 volts/mil.

7. An electrode for treating tissue, said electrode for use with an electrosurgical pencil connected to an electrosurgical generator comprising:
an electrically conductive rod having a distal end and a proximal end with an axis, said proximal end adapted to be connected to said electrosurgical pencil, said distal end terminating in a conducting electrode tip, said electrode tip comprising an active portion for serving as a source of radio frequency electromagnetic energy;
an insulating member situated about said rod and a predetermined portion of said electrode tip, said insulating member covering a predetermined portion of said active portion of said electrode tip.

8. A method of using an electrosurgical electrode to treat tissue comprising the steps of:
providing an electrosurgical electrode having an active portion thereof from which electromagnetic energy may emanate; and
interposing a dielectric material between said active portion of said electrode and the tissue to be treated.

9. A method according to claim 8 further comprising spacing said dielectric material a predetermined distance away from said active portion.
